(19) United States
(12) Patent Application Publication
(10) Pub. No.: US 2023/0331770 A1
(43) Pub. Date: Oct. 19, 2023

(54) ARGININE MODIFICATION AND CONJUGATION METHODS

(71) Applicant: Encodia, Inc., San Diego, CA (US)

(72) Inventors: Stephen VERESPY, III, San Diego, CA (US); Duc T. TRAN, San Diego, CA (US); Samantha BEASLEY, San Diego, CA (US)

(73) Assignee: Encodia, Inc., San Diego, CA (US)

(21) Appl. No.: 18/297,914

(22) Filed: Apr. 10, 2023

Related U.S. Application Data
(60) Provisional application No. 63/330,677, filed on Apr. 13, 2022.

(51) Int. Cl. C07K 1/107

(52) U.S. Cl. CPC C07K 1/1077

(57) ABSTRACT

Provided herein are methods for modifying an arginine residue to provide a reactive handle suitable for attaching the arginine residue to a label, linker, or other target molecule. The methods utilize an Arnold Salt to convert the guanidine group of the arginine residue into a formylpyrimidine, and are efficient and very selective. Provided herein are mild reaction conditions for this arginine modification, making it suitable for use with complex biomolecules like proteins. The methods can include one or more additional steps to attach the modified arginine to a linker for further manipulation or for connecting the arginine residue to a target compound. Provided methods are especially useful for covalently linking an arginine-containing peptide to a target molecule such as a polynucleotide.

### CROSS-REFERENCE TO RELATED APPLICATION

This application claims priority to U.S. Provisional Patent Application No. 63/330,677 filed Apr. 13, 2022, entitled “ARGININE MODIFICATION AND CONJUGATION METHODS,” which is herein incorporated by reference in its entirety for all purposes.

### REFERENCE TO AN ELECTRONIC SEQUENCE LISTING

The contents of the electronic sequence listing (2004200_SEQLIST.xml; Size: 9,484 bytes; and Date of Creation: Apr. 6, 2023) is herein incorporated by reference in its entirety.

### TECHNICAL FIELD

Provided herein are methods to selectively modify the side chain of the amino acid arginine or other guanidine compounds, using conditions sufficiently mild and specific for functionalizing a peptide in the presence of other biomolecules. In some embodiments, provided herein are selective and efficient methods to form conjugates of arginine-containing peptides, including conjugates connecting a peptide to a polynucleotide.

### BACKGROUND

Attaching linking groups, labels, markers, and fluorogenic probes to biological molecules such as peptides for the purpose of labeling the biological molecules or linking one biomolecule to another are vital tools to aid in understanding and utilizing complex biological systems. Ideally, such conjugates can be formed selectively and in good yield under conditions where the biological molecules are stable and functional, e.g., in a biological medium. Methods for attaching groups to biological molecules in complex aqueous media are known, but there remains a need for new methods complementary to existing ones and methods that are more selective and efficient than those known.

Methods for attaching a linker or cargo to proteins are especially important, and sometimes rely on reactivity of side chains of naturally occurring amino acids to attach a linker that can be used to connect the protein to a label, carrier, surface, or other entity. While such methods are known, few methods are available to selectively use the guanidine side chain of arginine for conjugation of proteins. Arginine is an appealing connection point because it is relatively common, and its guanidine group is not found in other common amino acids. There remains a need for conjugation methods and linking chemistries that are efficient, selective, and complementary to existing ones, and methods for conjugation to arginine would be suitable. The present invention addresses such needs with a method of modifying a guanidine group, which is present on the natural amino acid arginine, to provide a useful biorthogonal reactive handle and methods to use the new reactive handle for making conjugates.

### BRIEF SUMMARY

Provided herein are methods for using the Arnold Salt to transform a guanidine group into a formyl-substituted pyrimidine ring. The method provides reaction conditions for accomplishing the reaction under mild conditions, and is efficient when applied to the guanidine of the side chain of an arginine residue in a peptide. The formyl group introduced by this reaction serves as a reactive handle for further modification using methods and reagents known in the art. The reaction conditions for modifying the guanidine group are suitably mild for use on or in the presence of sensitive, complex biomolecules: the reaction can be conducted with high conversion under conditions where post-translational modifications of proteins remain intact.

In one aspect, the present disclosure provides a method to functionalize a guanidine, typically on the side chain of an amino acid residue, wherein the method comprises contacting the amino acid residue with an Arnold Salt under conditions that result in formation of a formyl-substituted pyrimidine of Formula (I) according to the following reaction:

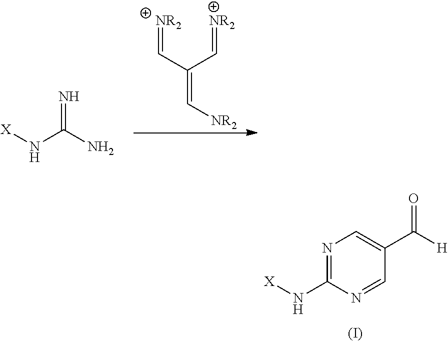

wherein:
   X represents the remainder of the side chain of the amino acid residue, and
   each R is independently selected from methyl, ethyl, and propyl, or two R on the same N can optionally be taken together with the nitrogen to which they are attached to form a 4-6 membered ring.

The method allows a user to modify a guanidine group, typically in a protein, to introduce a convenient reactive handle for further derivatization. The initial reaction introduces an aldehyde (formyl) group on a pyrimidine ring that can be used to further modify the compound bearing the guanidine, which is typically a peptide having at least one arginine residue or an arginine mimetic. Balakrishnan, et al., Chembiochem. 2012 Jan. 23; 13(2): 259-270. The aldehyde group can be reacted with certain complementary reactive handles, like alkoxyamines and alkyl hydrazines, to attach additional structure to the underlying peptide. The initial adduct formed by reaction of the aldehyde with an alkoxyamine or alkyl hydrazine is often reversible, but the initial adduct can be reduced to form a stable conjugate.

Provided herein is also a modified amino acid residue comprising a substructure of

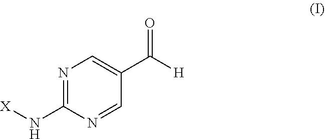

### Formula (I):

wherein X represents the side chain portion of the amino acid residue. The amino acid residue can be an arginine residue in a peptide or an arginine mimetic. The modified amino acid can be prepared using the method described above.

Provided herein is also a conjugate comprising the structure:

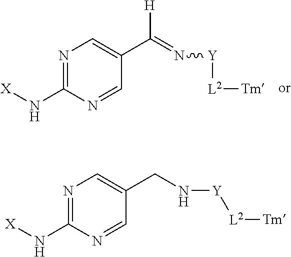

wherein X represents the side chain of an amino acid residue, typically an arginine, which may be a residue of a peptide;
L² is a linker;
Y is selected from a bond, O and NH; and
Tm′ represents a Target molecule.

Provided herein is also a method for attaching a peptide comprising at least one arginine residue to a target molecule, the method comprising the steps of:
(a) contacting the at least one arginine residue with an Arnold Salt under conditions that result in formation of a formyl-substituted pyrimidine of Formula (I):

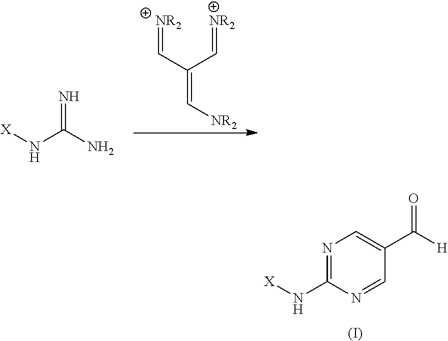

wherein:
   X represents the peptide connected through the at least one arginine residue, and each R is independently selected from methyl, ethyl, and propyl, or two R on the same N can optionally be taken together with the nitrogen to which they are attached to form a 4-6 membered ring; and
(b) contacting the formyl pyrimidine of Formula (I) with a compound that comprises the target molecule and an aldehyde-reactive reaction handle having an —NH₂ group, to form a conjugate of the formula:

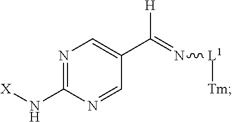

wherein X represents the peptide connected through the at least one arginine residue, L¹ represents a linker, and Tm represents the target molecule.

These conjugates have a peptide attached by a tether to a cargo compound, which can be a label, surface, or biomolecule. In a preferred embodiment, X represents the portion of an arginine side chain excluding the guanidine (which has been transformed into a pyrimidine ring) and Tm′ represents a polynucleotide.

These and other aspects and embodiments of the invention are represented and enabled by the detailed description and examples below.

### BRIEF DESCRIPTION OF THE DRAWINGS

FIG. 1 shows an exemplary schematic representation of modification of an arginine residue in a peptide using methods of the invention, providing a modified peptide comprising a 5-formyl pyrimidine formed by reaction of Arnold Salt with the side chain of an arginine. The NTAA and a lysine side chain were protected by acetylation before this step.

FIG. 2A and FIG. 2B illustrate evaluation of efficiencies of the Arnold Salt modification reaction under different reaction buffer conditions. Bicarb indicates 50 mM sodium bicarbonate buffer, pH=8.5; CBC indicates 0.1 M Carbonate/Bicarbonate buffer, pH 10.0; ACN indicates 50% acetonitrile co-solvent; plus indicates presence of 50 mM Arnold salt reagent; minus indicates absence of the Arnold salt reagent. FIG. 2A shows Percent depletion (depletion of the initial peptide peak based on the LC chromatographic data); and FIG. 2B shows Product intensity of the same reactions measured by ion counts in the LC-MS runs of the resulting products.

FIG. 3. Exemplary assessment of Arnold Salt arginine modification efficiencies at the proteome level in human serum samples. Peptides were purified from either serum samples (Serum) or depleted serum samples (DS); lysines of peptides were capped with NHS-DTB (DTB) or processed without capping (no DTB); and peptides were modified with the Arnold Salt reagent (AS) or processed without modification (no AS), as described in Example 2. Data show 60-70% conversion yield for the Arnold Salt-Arg conjugation in peptides across the human serum proteome as determined by MS/MS analysis.

FIG. 4A shows structure of Hydrazino nicotinamide (HNA) used in conjugation with Arnold salt. FIG. 4B shows the reaction of the product shown in FIG. 1 with an arylhydrazine attached to a target molecule, which is a DNA polynucleotide in the example. The polynucleotide is first acylated with an acylating agent shown in 4A to attach it to a 2-hydrazino-pyridinyl group, and the hydrazinyl group then condenses with the formyl group of the product shown in FIG. 1, producing a conjugate having a polynucleotide linked to a peptide.

FIG. 5A and FIG. 5B illustrate an exemplary assessment of On-bead Arnold salt arginine modification efficiencies as described in Example 4.

FIG. 5A shows product intensities measured by ion counts in the LC-MS runs of the resulting products treated with or without the Arnold salt reagent. Corresponding predicted masses of products are indicated. FIG. 5B shows product intensities measured by ion counts in the LC-MS runs of the resulting products treated with the Arnold salt reagent, and with or without HNA. Corresponding predicted masses of products are indicated.

FIG. 6 illustrates an exemplary synthesis scheme of bis(hexafluorophosphate) Arnold salt as described in Example 5.

FIG. 7 illustrates an exemplary workflow using arginine modification of the peptide immobilized on a bead with the Arnold Salt reagent to generate an immobilized peptide-DNA conjugate. After modification, the peptide-DNA conjugate is released from the bead, leaving the N-terminal amino acid (NTAA) of the peptide free.

### DETAILED DESCRIPTION

Non-limiting embodiments of the present invention will be described below by way of examples with reference to the accompanying figures, which are intended to illustrate some variations of the methods and compositions of the invention. For purposes of illustration, not every component is labeled in every figure, nor is every component of each embodiment of the invention shown where illustration is not necessary to allow those of ordinary skill in the art to understand the Figures and the invention.

The following description and examples are intended to illustrate and exemplify certain aspects and embodiments of the invention but are not intended to limit its scope. The scope of the various aspects of the invention is defined by the claims. Enumerated embodiments below describe some aspects of the invention.

Methods and compositions of the invention can be used for any suitable purpose. They are suitable for use in preparing samples for analysis and for preparing libraries of conjugates, which are useful in methods such as those disclosed in WO2017/192633 for analyzing peptides and tagging peptides with nucleic acids.

### DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

The term “comprising” as used herein takes its conventional meaning as used for patent purposes, and is considered an open transition. It thus refers to methods or compositions that include specified features and may optionally include ones. Thus a method ‘comprising’ steps (1) and (2) can optionally include additional steps such as (3) and (4), etc. Similarly, a composition comprising components (1) and (2) can optionally include additional components such as (3) and (4), etc.

The term “consisting of” as used herein takes its conventional meaning as used for patent purposes, and is considered a closed transition. It thus refers to methods or compositions that include specified steps or features and no additional ones.

The term “consisting essentially of” as used herein takes its conventional meaning as used for patent purposes, and refers to methods or compositions that include specified steps or features and may include additional steps or features that do not materially change the product or process described by the explicit steps or features.

An aspect of the invention described as ‘comprising’ certain features is intended to disclose and to include the aspects of the invention ‘consisting of’ and/or ‘consisting essentially of’ the recited features.

The term “alkyl” as used herein refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tertiary butyl, pentyl, isopentyl, hexyl, cyclopropylmethyl, etc.

Alkyl groups can be unsubstituted, or they can be substituted to the extent that such substitution makes sense chemically. Typical substituents include, but are not limited to, halo, ═O, ↑N—CN, ═N—OR^{a}, ═NR^{a}, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C1-C8 alkyl, C2-C8 heteroalkyl, C3-C8 heterocyclyl, C4-C10 heterocyclyclalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R^{a} is optionally substituted with halo, ═O, ═N—CN, ═N—OR^{b}, ═NR^{b}, OR^{b}, NR^{b}₂, SR^{b}, SO₂R^{b}, SO₂NR^{b}₂, NR^{b}SO₂R^{b}, NR^{b}CONR^{b}₂, NR^{b}COOR^{b}, NR^{b}COR^{b}, CN, COOR^{b}, CONR^{b}₂, OOCR^{b}, COR^{b}, and NO₂, wherein each R^{b} is independently H, C1-C8 alkyl, C2-C8 heteroalkyl, C3-C8 heterocyclyl, C4-C10 heterocyclyclalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where a substituent group contains two R^{a} or R^{b} groups on the same or adjacent atoms (e.g., —NR^{b}₂, or —NR^{b}—C(O) R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

The term “alkenyl” as used herein refers to an alkyl as defined above having at least two carbon atoms and at least one carbon-carbon double bond. Thus, particularly contemplated alkenyl groups include straight, branched, or cyclic alkenyl groups having two to ten carbon atoms (e.g., ethenyl, propenyl, butenyl, pentenyl, etc.) or 5-10 atoms for cyclic alkenyl groups. Alkenyl groups are optionally substituted by groups suitable for alkyl groups as set forth herein.

Similarly, the term “alkynyl” as used herein refers to an alkyl or alkenyl as defined above and having at least two (preferably three) carbon atoms and at least one carbon-carbon triple bond. Especially contemplated alkynyls include straight, branched, or cyclic alkynes having two to ten total carbon atoms (e.g., ethynyl, propynyl, butynyl, cyclopropylethynyl, etc.). Alkynyl groups are optionally substituted by groups suitable for alkyl groups as set forth herein.

The term “cycloalkyl” as used herein refers to a cyclic alkane (i.e., in which a chain of carbon atoms of a hydrocarbon forms a ring), preferably including three to eight carbon atoms. Thus, exemplary cycloalkanes include cyclopropyl, cyclobutyl, cyclopentyl, cyclohexyl, cycloheptyl, and cyclooctyl. Cycloalkyls also include one or two double bonds, which form the “cycloalkenyl” groups. Cycloalkyl groups are optionally substituted by groups suitable for alkyl groups as set forth herein.

The term “aryl” or “aromatic moiety” as used herein refers to an aromatic ring system, which may further include one or more non-carbon atoms. These are typically 5-6 membered isolated rings, or 8-10 membered bicyclic groups, and can be substituted. Thus, contemplated aryl groups include (e.g., phenyl, naphthyl, etc.) and pyridyl. Further contemplated aryl groups may be fused (i.e., covalently bound with 2 atoms on the first aromatic ring) with one or two 5- or 6-membered aryl or heterocyclic group, and are thus termed “fused aryl” or “fused aromatic”.

Aromatic groups containing one or more heteroatoms (typically N, O or S) as ring members can be referred to as heteroaryl or heteroaromatic groups. Typical heteroaromatic groups include monocyclic C5-C6 aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms.

As also used herein, the terms “heterocycle”, “cycloheteroalkyl”, and “heterocyclic moieties” are used interchangeably herein and refer to any compound in which a plurality of atoms form a ring via a plurality of covalent bonds, wherein the ring includes at least one atom other than a carbon atom as a ring member. Particularly contemplated heterocyclic rings include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Typically these rings contain 0-1 oxygen or sulfur atoms, at least one and typically 2-3 carbon atoms, and up to four nitrogen atoms as ring members. Further contemplated heterocycles may be fused (i.e., covalently bound with two atoms on the first heterocyclic ring) to one or two carbocyclic rings or heterocycles, and are thus termed “fused heterocycle” or “fused heterocyclic ring” or “fused heterocyclic moieties” as used herein. Where the ring is aromatic, these can be referred to herein as ‘heteroaryl’ or heteroaromatic groups.

Heterocyclic groups that are not aromatic can be substituted with groups suitable for alkyl group substituents, as set forth above.

Aryl and heteroaryl groups can be substituted where permitted. Suitable substituents include, but are not limited to, halo, —OR^{a}, —NR^{a}₂, —SR^{a}, —SO₂R^{a}, —SO₂NR^{a}₂, —NR^{a}SO2R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C1-C8 alkyl, C2-C8 heteroalkyl, C3-C8 heterocyclyl, C4-C10 heterocyclyclalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R^{a} is optionally substituted with halo, ═O, ═N—CN, =N—OR^{b}, =NR^{b}, OR^{b}, NR^{b}₂, SR^{b}, SO₂R^{b}, SO₂NR^{b}₂, NR^{b}SO₂R^{b}, NR^{b}CONR^{b}₂, NR^{b}COOR^{b}, NR^{b}COR^{b}, CN, COOR^{b}, CONR^{b}₂, OOCR^{b}, COR^{b}, and NO₂, wherein each R^{b} is independently H, C1-C8 alkyl, C2-C8 heteroalkyl, C3-C8 heterocyclyl, C4-C10 heterocyclyclalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where a substituent group contains two R^{a} or R^{b} groups on the same or adjacent atoms (e.g., —NR^{b}₂, or —NR^{b}—C(O) R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member. As also used herein, the terms “guanidine” refers to a molecular substructure that has this formula:

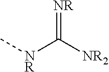

where the dashed bond indicates where the guanidine group is attached to a base moiety such as an alkyl group or amino acid side-chain. The guanidine group serves as a starting component in reaction so the invention, where it is preferably unsubstituted, which means each R represents hydrogen (H). A preferred embodiment of a guanidine for the methods and compositions herein is the guanidine side chain of an amino acid, typically arginine or an arginine-mimetic, which may be alone (optionally in protected form) or a residue in a peptide. Examples of Arginine Mimetics:

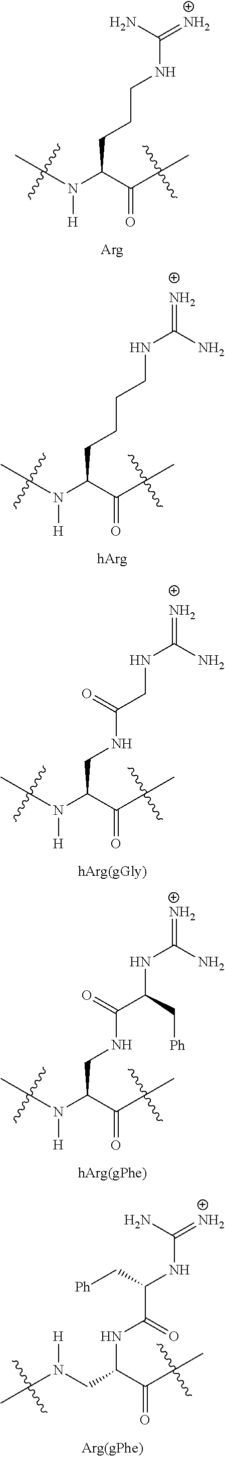 [SMILES: *N[C@@H](CCCNC(N)=[NH2+])C(*)=O ; *N[C@@H](CCCCNC(N)=[NH2+])C(*)=O ; *N[C@@H](CNC(=O)CNC(N)=[NH2+])C(*)=O ; *N[C@@H](CNC(=O)[C@H](Cc1ccccc1)NC(N)=[NH2+])C(*)=O ; *NC[C@H](NC(=O)[C@H](Cc1ccccc1)NC(N)=[NH2+])C(*)=O]

“Arnold Salt” as used herein refers to the following cationic species:

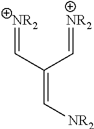

where each R is independently selected from methyl, ethyl, and propyl, or alternatively two R groups on the same nitrogen, taken together with the nitrogen atom, form a 4-6 membered heterocyclic ring. A preferred Arnold Salt is the readily accessible one wherein each R is methyl, referred to as 2-dimethylaminomethylene-1,3-bis(dimethylimonio)propane.

The cation of the Arnold Salt is accompanied by anionic counterions, typically selected from Br₃⁻, ClO₄⁻, PF₆⁻, and BF₄⁻. The nature of the counterion has little impact on the reaction and is thus selected based on convenience or availability. A preferred counterion is PF₆⁻, particularly when each R is methyl, because the bis-(hexafluorophosphate) salt of 2-dimethylaminomethylene-1,3-bis(dimethylimonio)propane is readily synthesized and purified. For convenience, this particular salt is sometimes referred to as ‘the’ Arnold Salt.

The term “aryloxy” as used herein refers to an aryl group connecting to an oxygen atom, wherein the aryl group may be further substituted. For example, suitable aryloxy groups include phenyloxy, pyrdinyloxy, etc. Similarly, the term “arylthio” as used herein refers to an aryl group connecting to a sulfur atom, wherein the aryl group may be further substituted. For example, suitable arylthio groups include phenylthio, etc.

The hydrocarbon portion of each alkoxy, alkylthio, alkylamino, and aryloxy, etc. can be substituted as appropriate for the relevant hydrocarbon moiety.

The term “halogen” as used herein refers to fluorine, chlorine, bromine and iodine. Where present as a substituent group, halogen or halo typically refers to F or Cl or Br, more typically F or Cl.

The term “haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

The term “haloalkoxy” refers to the group alkyl-O- wherein one or more hydrogen atoms on the alkyl group have been substituted with a halo group and include, by way of examples, groups such as trifluoromethoxy, and the like.

The term “sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein

The term “aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

The term “acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

It should further be recognized that all of the above-defined groups may further be substituted with one or more substituents, which may in turn be substituted with hydroxy, amino, cyano, C₁-C₄ alkyl, halo, or C₁-C₄ haloalkyl. For example, a hydrogen atom in an alkyl or aryl can be replaced by an amino, halo or C₁₋₄ haloalkyl or alkyl group.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃⁺), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, POSH, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, compounds arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

As used herein, the term ‘bioorthogonal reactive handle’ refers to a reactive moiety that is stable in typical biological media and systems, and reacts specifically with appropriate non-biological complementary reactive groups under mild conditions that do not damage the biological system. Examples of bioorthogonal reactive handles include tetrazines, which can react with complementary reactive groups including strained alkenes and alkynes such as cyclopropenes, trans-cyclooctene, cyclooctyne, and the like; alkyl azides, which take part in ‘click’ reactions with complementary reactive groups including terminal alkynes and alkenes; and phosphines and azides, which can take part in Staudinger ligation reactions to form amide bonds. Examples of bioorthogonal reactive handles and strategies for using them are well known in the art. See e.g., C. P. Ramil, et al., Chem. Commun. 2013, vol. 49, 11007-11022; M. F. Debets, et al., Org. Biomol. Chem. 2013, vol. 11, 6439.

As used herein, the term “inverse diene” refers to an electron-poor diene capable of reacting with an electron-rich multiple bond in an inverse-electron demand Diels-Alder reaction, such as a 1,2,4,5-tetrazine.

As used herein, the term “detectable label” refers to any labels that can be utilized and are compatible with the provided peptide analysis assay format and include, but are not limited to, a bioluminescent label, a biotin/avidin label, a chemiluminescent label, a chromophore, a coenzyme, a dye, an electro active group, an electrochemiluminescent label, an enzymatic label, a fluorescent label, a latex particle, a magnetic particle, a metal, a metal chelate, a phosphorescent dye, a protein label, a radioactive element or moiety, and a stable radical. Examples include, but are not limited to, 1,4,7,10-Tetraazacyclododecane-1,4,7,10-tetraacetic acid (DOTA), desthiobiotin, TAMRA, fluorogenic labels, isobaric mass tags, and 2-formylphenylboronic acid.

The term ‘conjugation reagent’ as used herein refers to an organic moiety that can be used to, or is used to, connect (link) two organic groups by reacting with features of the two organic groups to form covalent linkages. Examples include connecting a target compound to at least one other molecule, such as a reactive handle, functional group, label, binding group, tag, or other target molecule. A conjugation reagent can comprise one or more various groups such as reactive handles and/or detectable labels.

The term “modify” or “modified” as used herein refers to a chemical transformation that covalently changes a molecule or molecular fragment being described. For example, a guanidine is ‘modified’ by reaction with Arnold Salt as described herein to form a 2-amino-(5-formyl)pyrimidine; when this transformation is done to a peptide comprising a guanidine, the peptide may then be described as a modified peptide.

The term ‘acylated NH₂’ as used herein refers to an NH₂ group that is attached to a C═X group, where X is O, S or NR, where R is H or C₁₋₄ alkyl. Acylated NH₂ groups include guanidine, urea, thiourea, and amidine groups.

The term ‘aqueous medium’ as used herein refers to a solvent or solvent mixture that comprises a significant fraction of water, i.e., at least 25% water by volume. The aqueous medium can include one or more co-solvents, including organic co-solvents such as acetonitrile, DMSO, DMF, DMA, NMP, TMU, cyrene, sulfolane, 2-methyl THF, limonene, 1,3-dimethylpyridone, THF, dioxane, DME, alcohols such as methanol, ethanol, isopropanol, t-butanol, n-butanol, ethylene glycol, propylene glycol, polyethylene glycol, and the like. In some embodiments, the aqueous medium comprises 1-75% organic cosolvent such as those just named, or a mixture of two or more of such cosolvents. In some embodiments, the aqueous medium comprises 10-65% organic cosolvent(s). In some embodiments, the aqueous medium comprises 30-60% organic co-solvents, or 40-60% organic co-solvent, or about 50% organic cosolvent

As used herein, the term “reactive handle” refers to a moiety on a first molecule that can be caused to react with a second molecule having a complementary ‘reactive handle’ to form a covalent bond between the first molecule and the second molecule. Typical complementary pairs of reactive handles include functional groups such as carboxylate groups and amines, which can react with each other to form amides; thiols and alkylating reagents that can be reacted to form thioethers; thiols and maleimides that can be reacted to form thiosuccinimides; strained alkenes or alkynes and 1,3-dipoles such as azides that can react via cycloaddition reactions, e.g., copper-free click chemistry; and tetrazines that can react via inverse-electron demand Diels-Alder chemistry with electron rich or strained alkenes and alkynes.

For each reactive handle, there is a complementary reactive handle that will react with it to form a covalent linkage. A ‘complementary reactive handle’ as used herein refers to one of a pair of reactive handles that react with each other. Many examples are known, see e.g., M. F. Debets, et al., Org. Biomol. Chem. 2013, vol. 11, 6439. For example, an alkyl azide is a complementary reactive handle that can be used with a terminal alkyne: the alkyl azide and terminal alkyne can react to form a triazole ring, and the reaction can be used to connect two compounds together. Tetrazines are well known reactive handles: they can react in ‘tetrazine ligation’ reactions with a variety of complementary reactive handles, e.g., norbornenes, cyclooctynes, and trans-cyclooctenes:

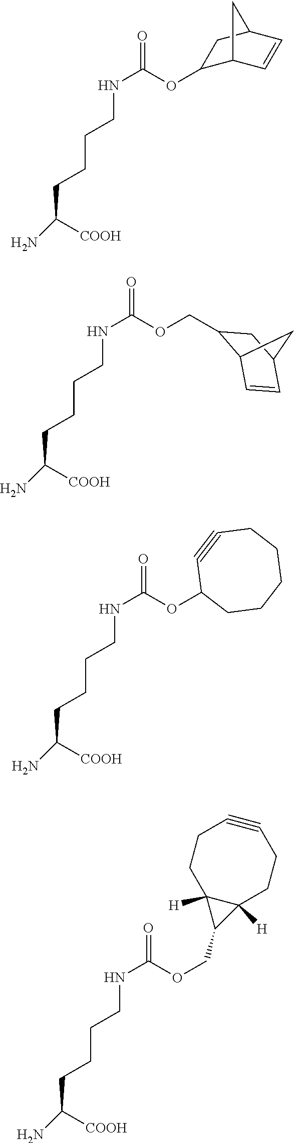 [SMILES: N[C@@H](CCCCNC(=O)OC1CC2C=CC1C2)C(=O)O ; N[C@@H](CCCCNC(=O)OCC1CC2C=CC1C2)C(=O)O ; N[C@@H](CCCCNC(=O)OC1C#CCCCCC1)C(=O)O ; N[C@@H](CCCCNC(=O)OC[C@@H]1[C@@H]2CCC#CCC[C@@H]21)C(=O)O]

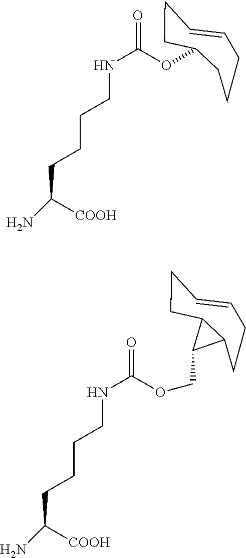

C. P. Ramil, et al., Chem. Commun. 2013, vol. 49, 11007-11022.

As used herein, ‘click chemistry’ refers to reactions and reactants that are commonly used in biological systems, and are useful as reactive handles in the conjugation reagents and methods of the invention. Click chemistry reactive handles include reactants for inverse-electron demand Diels-Alder reactions, such as tetrazines, which react efficiently with a variety of activated alkene and alkyne groups such as cyclopropenes and trans-cyclooctene, and reactants for [3+2] cycloadditions, such as azide which reacts efficiently with an electron rich alkene or alkyne. Tetrazines are well known reactive handles for attaching fluorogenic probes to biomolecules such as peptides to enable visualization of target biomolecules in cells. Y. Lee, et al., J. Am. Chem. Soc. 2018, 140, 974-983. Tetrazine rings are stable in biological media, and react with specific reaction partners under mild conditions, so they are very useful for attaching a probe to a target with good selectivity.

“Bioorthogonal” reactive handles are reactive handles that can be used in biological systems, i.e., in aqueous media, and that are generally not reactive toward common functional groups in the biological system, so they can be used to manipulate biological compounds selectively, without interference from the biomolecule components. Bioorthogonal chemistry is well known in the art: suitable functional groups for bioorthogonal chemistry include ketones, aldehydes, hydrazides, alkoxyamines, azides, terminal alkynes, phosphines, nitrones, nitrile oxides, diazo compounds, tetrazines, tetrazoles, quadrocyclanes, alkenes, iodobenzenes, transcyclooctenes, cyclooctynes, norbornenes, cyclopropenes, vinyls, isonitriles, and cycloaddition reactants. M. F. Debets, et al., Org. Biomol. Chem. 2013, vol. 11, 6439. Examples include click chemistry, particularly copper-free click chemistry, which uses cycloaddition reactants like cyclooctyne that react efficiently with alkyl azides; and inverse-electron demand Diels-Alder chemistries such as tetrazines, which react with strained alkenes or alkynes like cyclopropene and trans-cyclooctene as well as strained alkynes like cyclooctynes. Useful cyclooctynes include:

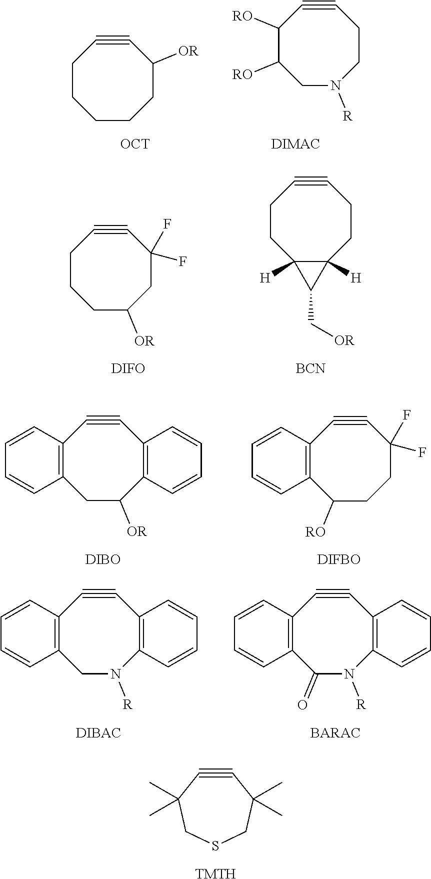

‘R’ in these structures indicates where the cyclooctyne compound can be attached to a target molecule or conjugation reagent, etc. TMTH is actually a 7-membered ring, but the C—S bonds are longer than C—C bonds, so the ring strain is similar to that of a cyclooctyne. C. P. Ramil, et al., Chem. Commun. 2013, vol. 49, 11007-11022.

As used herein, the term ‘leaving group’ refers to a moiety that is readily displaced by reaction with a complementary reactant, which is often a nucleophile. In some examples herein, the leaving group is on an acyl carbon, e.g., R—C(═O)LG, where LG is a displaceable leaving group; such acyl groups can react with a nucleophile, where the leaving group is replaced by the nucleophile. Examples of leaving groups for such acyl groups include, but are not limited to, halo, CN, azide, acyl groups such as pivaloate, alkoxyacyloxy groups such as isobutoxy-carbonyl-O, imidazole, triazole, anhydride, sulfonyl, hydrazide, sulfonylhydrazide, azobenzotriazole, pentafluorophenol, dinitrophenol, -O-benzotriazole, ethyl cyanohydroxyiminoacetate, activated alkoxy groups such as trifluorethoxy and trichloroethoxy, and —OC(O)OR where R is a C₁₋₈ alkyl.

As used herein, the term “linking group” or “linker” refers to a stable organic tether for connecting two (or more than two) chemical groups together. If not otherwise specified, the linking group contains up to 100 carbon atoms and up to 24 heteroatoms selected from N, O and S, and is optionally substituted with 1-3 groups selected from C₁₋₃ alkoxy, oxo, CN, and halo. In some embodiments, the linking group comprises up to 50 carbon atoms and up to 20 heteroatoms. In other embodiments, the organic linking group comprises up to 20 carbon atoms and up to 7 heteroatoms.

As used herein, the term “peptide” encompasses peptides, polypeptides and proteins, and refers to a molecule comprising a chain of three or more amino acid residues joined by peptide bonds. In preferred embodiments, peptide contains from 4 to 40 amino acid residues. In general terms, a peptide having more than 20-30 amino acids is commonly referred to as a polypeptide, and one having more than 50 amino acids is commonly referred to as a protein. The amino acids of the peptide are most typically L-amino acids, but may also be D-amino acids, modified amino acids, amino acid analogs, amino acid mimetics, or any combination thereof. Peptides may be naturally occurring, synthetically produced, or recombinantly expressed. Peptides may also comprise additional groups modifying the amino acid chain, for example, functional groups added via post-translational modification.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “solid support” refers to any solid material, including porous and non-porous materials, to which a macromolecule (e.g., peptide) can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a nitrocellulose membrane, a nitrocellulose-based polymer surface, a polymer matrix, a nanoparticle, or a microsphere. Materials for a solid support include but are not limited to acrylamide, agarose, cellulose, nitrocellulose, glass, gold, quartz, polystyrene, polyethylene vinyl acetate, polypropylene, polymethacrylate, polyethylene, polyethylene oxide, polysilicates, polycarbonates, Teflon, fluorocarbons, nylon, silicon rubber, polyanhydrides, polyglycolic acid, polyactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a paramagnetic bead, a glass bead, or a controlled pore bead. A bead may be spherical or an irregularly shaped. A bead's size may range from nanometers, e.g.. 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding.

The term “sequence identity” is a measure of identity between peptides at the amino acid level, and a measure of identity between nucleic acids at nucleotide level. The peptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The terms “corresponding to position(s)” or “position(s) . . . with reference to position(s)” of or within a peptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the peptide upon alignment with the disclosed sequence using a standard alignment algorithm, such as the BLAST algorithm (NCBI). One skilled in the art can identify any given amino acid residue in a given peptide at a position corresponding to a particular position of a reference sequence, such as set forth in the Sequence Listing, by performing alignment of the peptide sequence with the reference sequence (for example, by using BLASTP publicly available through the NCBI website), matching the corresponding position of the reference sequence with the position in peptide sequence and thus identifying the amino acid residue within the peptide.

The term “peptide bond” as used herein refers to a chemical bond formed between two molecules (such as two amino acids) when the carboxyl group of one molecule reacts with the amino group of the other molecule, releasing a water molecule (H2O).

The term “unmodified” (also “wild-type” or “native”) as used herein is used in connection with biological materials such as nucleic acid molecules and proteins (e.g., cleavase), refers to those which are found in nature and not modified by human intervention.

The term “modified” or “engineered” (or “variant”, or “mutant”) as used in reference to nucleic acid molecules and protein molecules, e.g., an engineered binder or engineered cleavase enzyme, implies that such molecules are created by human intervention and/or they are non-naturally occurring. The variant, mutant or engineered binder or cleavase is a peptide having an altered amino acid sequence, relative to an unmodified or wild-type protein, such as starting scaffold, or a portion thereof. An engineered enzyme is a peptide which differs from a wild-type enzyme scaffold sequence, or a portion thereof, by one or more amino acid substitutions, deletions, additions, or combinations thereof. An engineered binder generally exhibits at least 70%, 80%, 85%, 86%, 87%, 88%, 89%, 90%, 91%, 92%, 93%, 94%, 95%, 96%, 97%, 98%, 99% or more sequence identity to a corresponding wild-type starting protein scaffold. Non-naturally occurring amino acids as well as naturally occurring amino acids are included within the scope of permissible substitutions or additions.

In some embodiments, variants of an engineered binder or cleavase displaying only non-substantial or negligible differences in structure can be generated by making conservative amino acid substitutions in the engineered binder or cleavase. By doing this, further engineered binder variants that comprise a sequence having at least 90% (90%, 91%, 92%, 93%, 94%, 95%, 96%, 97%, 98%, and 99%) sequence identity with the initial engineered binder sequences can be generated, retaining at least one functional activity of the engineered binder, e.g. ability to specifically bind to the N-terminally modified target peptide. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The terms “specifically binding” and “specifically recognizing” are used interchangeably herein and generally refer to an engineered binder that binds to a cognate target peptide or a portion thereof more readily than it would bind to a random, non-cognate peptide. The term “specificity” is used herein to qualify the relative affinity by which an engineered binder binds to a cognate target peptide. Specific binding typically means that an engineered binder binds to a cognate target peptide at least twice more likely that to a random, non-cognate peptide (a 2:1 ratio of specific to non-specific binding). Non-specific binding refers to background binding, and is the amount of signal that is produced in a binding assay between an engineered binder and an N-terminally modified target peptide when the modified NTAA residue cognate for the engineered binder is not present at the N-terminus of the target peptide. In some embodiments, specific binding refers to binding between an engineered binder and an N-terminally modified target peptide with a dissociation constant (Kd) of 200 nM or less.

In some embodiments, binding specificity between an engineered binder and an N-terminally modified target peptide is predominantly or substantially determined by interaction between the engineered binder and the modified NTAA residue of the N-terminally modified target peptide, which means that there is only minimal or no interaction between the engineered binder and the penultimate terminal amino acid residue (P2) of the target peptide, as well as other residues of the target peptide. In some embodiments, the engineered binder binds with at least 5 fold higher binding affinity to the modified NTAA residue of the target peptide than to any other region of the target peptide. In some embodiments, the engineered binder has a substrate binding pocket with certain size and/or geometry matching the size and/or geometry of the modified NTAA residue of the N-terminally modified target peptide, to which the engineered binder specifically binds to. In such embodiments, the modified NTAA residue occupies a volume encompassing a substrate binding pocket of the engineered binder that effectively precludes the P2 residue of the target peptide from entering into the substrate binding pocket or interacting with affinity-determining residues of the engineered binder. In some embodiments, the engineered binder specifically binds to N-terminally modified target peptides, wherein the target peptides share the same modified NTAA residue that interacts with the engineered binder, but have different P2 residues. In some embodiments, the engineered binder is capable of specifically binding to each N-terminally modified target peptide from a plurality of N-terminally modified target peptides, wherein the plurality of N-terminally modified target peptides contains at least 3, at least 5, or at least 10 N-terminally modified target peptides that were modified with the same N-terminal modifier agent, have the same modified NTAA residue, and have different P2 residues. Thus, in preferred embodiments, the engineered binder possesses binding affinity towards the modified NTAA residue of the N-terminally modified target peptide, but has little or no affinity towards P2 or other residues of the target peptide.

As used herein, “nucleic acid sequencing” means the determination of the order of nucleotides in a nucleic acid molecule or a sample of nucleic acid molecules. Similarly, “peptide sequencing” means the determination of the identity and order of at least a portion of amino acids in the peptide molecule or in a sample of peptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times)—this depth of coverage is referred to as “deep sequencing.” Examples of high throughput nucleic acid sequencing technology include platforms provided by Illumina, BGI, Qiagen, Thermo-Fisher, and Roche.

As used herein, “analyzing” the peptide means to quantify, characterize, distinguish, or a combination thereof, all or a portion of the components of the peptide (e.g., partial identification of one or more amino acid residues (contiguous or non-continuous) of the peptide). For example, partial identification of amino acid residues in the peptide sequence can identify an amino acid in the protein as belonging to a subset of possible amino acids. Analysis typically begins with analysis of then NTAA, and then proceeds to the next amino acid of the peptide (i.e., n-1, n-₂, n-3, and so forth). This is accomplished by cleavage of the n NTAA, thereby converting the n-1 amino acid of the peptide to an N-terminal amino acid (referred to herein as the “n-1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The terminal amino acid at one end of the peptide chain that has a free amino group is referred to herein as the “N-terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the nth amino acid (also referred to herein as the “n NTAA”). Using this nomenclature, the next amino acid is the n-1 amino acid, then the n-2 amino acid, and so on down the length of the peptide from the N-terminal end to C-terminal end. In certain embodiments, an NTAA, CTAA, or both may be modified or labeled with a chemical moiety.

As used herein, the term “coding tag” refers to a nucleic acid molecule of about 2 bases to about 100 bases, including any integer including 2 and 100 and in between, that comprises identifying information for its associated binding agent. As used herein, the term “recording tag” refers to a nucleic acid molecule of about 2 bases to about 100 bases that optionally comprises identifying information for a peptide to which it is associated. In certain embodiments, after a binding agent binds a peptide, information from a coding tag linked to a binding agent can be transferred to the recording tag associated with the peptide while the binding agent is bound to the peptide.

The compounds and substructures described herein include stable tautomers of the depicted structure as well as the structure depicted.

The present invention uses reagents and methods that selectively react with and modify a guanidine group, especially the guanidine of an arginine residue in a protein. The invention provides reactions useful to modify a protein that comprises at least one guanidine group, by transforming the guanidine of the arginine side chain to form a formyl pyrimidine, and reaction conditions to perform this transformation under conditions mild enough to be compatible with proteins, including ones with post-translational modifications. It further provides methods to attach a linker or cargo molecule to a functionalized protein comprising the modified guanidine, as well as compositions that comprise the functionalized protein and protein conjugates having a linker that comprises a product formed from the formyl pyrimidine.

The following enumerated embodiments are representative of the invention:
1. A method to functionalize a guanidine on the side chain of an amino acid residue, wherein the method comprises contacting the amino acid residue with an Arnold Salt under conditions that result in formation of a formyl-substituted pyrimidine of Formula (I):

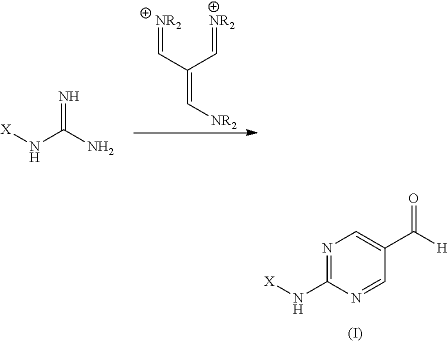

wherein:
   X represents the remainder of the side chain of the amino acid residue, and
   each R is independently selected from methyl, ethyl, and propyl, or two R on the same N can optionally be taken together with the nitrogen to which they are attached to form a 4-6 membered ring. It is understood that an anionic counterion accompanies an Arnold Salt, but is not involved in the chemical transformation or incorporated into the product of interest.
2. The method of embodiment 1, wherein the amino acid residue is an arginine in a peptide.
3. The method of embodiment 1 or ₂, wherein the arginine residue is contacted with an Arnold Salt in a medium comprising an aqueous buffer.
4. The method of embodiment 3, wherein the medium comprises an organic cosolvent.
5. The method of embodiment 4, wherein the organic cosolvent is acetonitrile.
6. The method of any one of the preceding embodiments, wherein the arginine residue is contacted with an Arnold Salt in a medium at a temperature between 40° C. and 80° C.
7. The method of embodiment 6, wherein the medium has a pH between 8 and 12.
8. The method of any one of the preceding embodiments, wherein at least 50% of the arginine residue in a sample is modified to form the formyl pyrimidine of Formula (I).
9. The method of any one of the preceding embodiments, wherein the Arnold Salt is associated with a counterion selected from Br₃⁻, ClO₄⁻, PF₆⁻, and BF₄⁻.
10. The method of any one of the preceding embodiments, wherein each R is methyl.
11. The method of any one of the preceding embodiments, wherein the peptide is joined to a solid support.
12. The method of any one of the preceding embodiments, which further comprises a step of contacting the formyl pyrimidine of Formula (I) with a target compound that comprises an aldehyde-reactive reaction handle having an —NH₂ group, to form a conjugate of the formula:

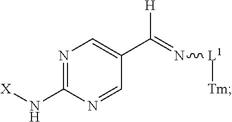

wherein X represents the alkyl portion of the side chain of an arginine residue,
L¹ represents a linker, and
Tm represents the target molecule.
13. The method of embodiment 12, wherein the conjugate is of the formula:

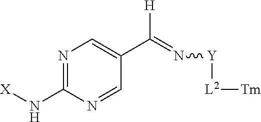

wherein X represents the alkyl portion of the side chain of the arginine residue,
Y is O or NH,
L² represents a linker; and
Tm represents the target molecule.
14. The method of embodiment 13, wherein the method further comprises a step of contacting the conjugate with a reducing agent to provide a conjugate of the formula:

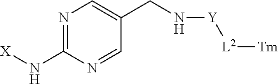

wherein X represents the alkyl portion of the side chain of the arginine residue,
Y is O or NH, and
Tm represents the target molecule.
15. The method of any one of embodiments 12-14, wherein X represents the alkyl portion of the side chain of an arginine residue in a peptide.
16. The method of embodiment 15, wherein Tm represents a polynucleotide.
17. A modified amino acid residue comprising a substructure of Formula (I):

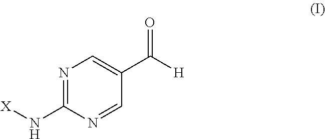

wherein X represents the side chain portion of the amino acid residue.
18. The modified arginine residue of embodiment 17, wherein the amino acid residue is an arginine in a peptide.
19. The modified arginine residue of embodiment 18, wherein the peptide is joined to a solid support.
20. A conjugate comprising the structure:

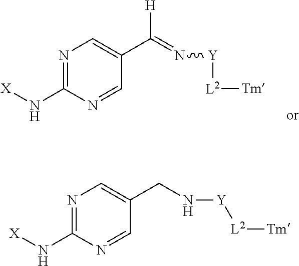

wherein X represents a side chain of an amino acid residue,
L2 is a linker;
Y is selected from a bond, O and NH; and
Tm′ represents a target molecule.
21. The conjugate of embodiment 20, wherein X represents the side chain of an arginine residue in a peptide.
22. The conjugate of embodiment 20 or 21, wherein Tm′ represents a polynucleotide.
23. The conjugate of embodiment 21, wherein the peptide is joined to a solid support.
24. The conjugate of any one of embodiments 20-23, wherein Y is —O— or —NH—.
25. A method of attaching a peptide comprising at least one arginine residue to a target molecule, the method comprising the steps of:
   (a) contacting the at least one arginine residue with an Arnold Salt under conditions that result in formation of a formyl-substituted pyrimidine of Formula (I):

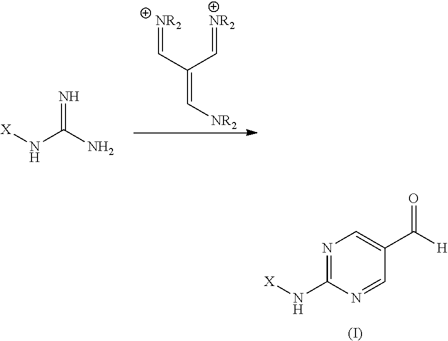

wherein:
   X represents the peptide connected through the at least one arginine residue, and each R is independently selected from methyl, ethyl, and propyl, or two R on the same N can optionally be taken together with the nitrogen to which they are attached to form a 4-6 membered ring; and
(b) contacting the formyl pyrimidine of Formula (I) with a compound that comprises the target molecule and an aldehyde-reactive reaction handle having an —NH₂ group, to form a conjugate of the formula:

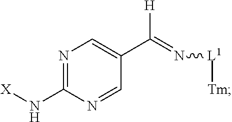

wherein X represents the peptide connected through the at least one arginine residue, L¹ represents a linker, and Tm represents the target molecule.
26. The method of embodiment 25, wherein the target molecule comprises a polynucleotide.
27. The method of embodiment 25 or 26, wherein the peptide is joined to a solid support.
28. The method of any one of embodiments 25-27, wherein the conjugate is of the formula:

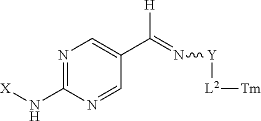

wherein X represents the peptide connected through the at least one arginine residue, Y is —O— or —NH—, L² represents a linker; and Tm represents the target molecule.
29. The method of embodiment 28, wherein the method further comprises a step of contacting the conjugate with a reducing agent to provide a conjugate of the formula:

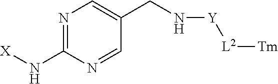

wherein X represents the peptide connected through the at least one arginine residue, Y is —O— or —NH—, and Tm represents the target molecule.

Suitable compounds for use in the methods and compositions of the invention include peptides, carbohydrates, nucleic acids, and other macromolecules that comprise at least one unsubstituted terminal guanidine group. Typically, the starting compound comprises an arginine residue, and frequently it is a peptide containing at least one arginine residue. Alternatively, a non-natural amino acid containing guanidine group may be utilized as the starting compound for the disclosed reactions. The method comprises contacting this starting compound with an Arnold Salt under conditions that promote reaction of the guanidine with the Arnold Salt, thereby modifying the guanidine moiety to form a 5-formyl pyrimidine ring, with the starting compound attached to the pyrimidine at its 2-position as illustrated here in Scheme (I):

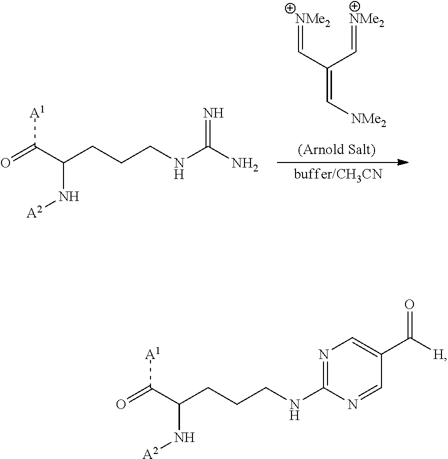

where A¹ can be an alkoxy group or the alpha-nitrogen of another amino acid residue, either alone or comprised in a polypeptide, and A² can be a nitrogen protecting group (e.g., acetyl, t-butoxycarbonyl, or the like) or the carbonyl of another amino acid residue. The modified arginine produced in this reaction has an aldehyde group that can selectively react with certain bioorthogonal reactive handles, and is thus useful for further derivatizing the compound or conjugating it to a target molecule.

Provided herein is also a method for attaching a peptide comprising at least one arginine residue to a target molecule, the method comprising the steps of:
(a) contacting the at least one arginine residue with an Arnold Salt under conditions that result in formation of a formyl-substituted pyrimidine of Formula (I):

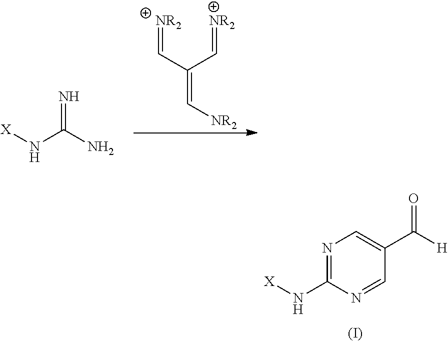

wherein:
   X represents the peptide connected through the at least one arginine residue, and each R is independently selected from methyl, ethyl, and propyl, or two R on the same N can optionally be taken together with the nitrogen to which they are attached to form a 4-6 membered ring; and
(b) contacting the formyl pyrimidine of Formula (I) with a compound that comprises the target molecule and an aldehyde-reactive reaction handle having an —NH₂ group, to form a conjugate of the formula:

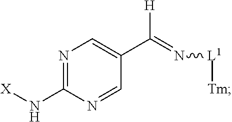

wherein X represents the peptide connected through the at least one arginine residue, L¹ represents a linker, and Tm represents the target molecule.

In some embodiments, the peptide is joined to a solid support. Various reactions may be used to attach the peptide to a solid support (e.g., a bead). In preferred embodiments, bioorthogonal reactions are utilized that offer fast kinetics and permissible reaction conditions. The peptide may be attached directly or indirectly to the support. Exemplary reactions include click chemistry reactions, such as the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO).

In certain embodiments, the peptide can be immobilized to a support by an affinity capture reagent. In one embodiment, the peptide macromolecule is attached to a bait nucleic acid which hybridizes to a capture nucleic acid which attached to the support, or comprises a reactive coupling moiety for attaching to the support. In some embodiments, the peptide can be immobilized to a support through nucleic acid hybridization as disclosed in US 2022/0049246 A1, incorporated herein by reference.

In some embodiments, the target molecule comprises a polynucleotide. In other embodiments, the target molecule does not comprise a polynucleotide. In some embodiments, the target molecule comprises a peptide molecule. In some embodiments, the target molecule comprises a macromolecule. In some embodiments, the target molecule comprises a label.

The reaction conditions for reaction of the arginine with Arnold Salt are mild: the reaction occurs at a useful rate in aqueous buffer at a pH of about 8-12, about 8-11, about 9-11, or about 9-12. The buffer can be bicarbonate or a carbonate/bicarbonate mixture, and other buffers such as phosphate, TRIS, borate, and the like can also be used.

An organic co-solvent that is miscible with water can accelerate the reaction: acetonitrile is one suitable co-solvent; other options for the co-solvent include dioxane, THF, dimethoxyethane, and alcohols such as methanol, ethanol or isopropanol. In some embodiments, the aqueous medium comprises about 40-60% organic co-solvent such as acetonitrile. An aqueous medium comprising about 50% acetonitrile as co-solvent is suitable.

In some preferred embodiments, at least 50% of the arginine residues in a sample are modified to form the formyl pyrimidine shown in Scheme (I). In some preferred embodiments, conversion of an arginine to a modified arginine of Scheme (I) was about 50%, 60%, 70%, 80%, or 90% complete. For example, when using a pH 10 Carbonate-bicarbonate aqueous buffer without organic co-solvents and a reaction temperature of about 60° C., conversion of an arginine to a modified arginine of Scheme(I) was about 60-70% complete within several hours. In another example, when using a pH 10 buffer and acetonitrile (about 50%) as co-solvent and a reaction temperature of about 60° C., conversion of an arginine to a modified arginine of Scheme(I) was about 90% complete within several hours.

The formyl pyrimidine of Scheme(I) is stable enough to be produced in aqueous solution, and can be isolated if desired. It is sufficiently reactive to undergo selective, efficient reaction with, e.g., an alkoxy or aryloxy amine or an alkyl or aryl hydrazine: condensation with these reagents can be used to append a further molecular structure to the modified amino acid residue. The further molecular structure can comprise a reactive handle for further modifications, such as a click-chemistry reactive handle, or it can comprise a target molecule that becomes covalently attached to the underlying protein.

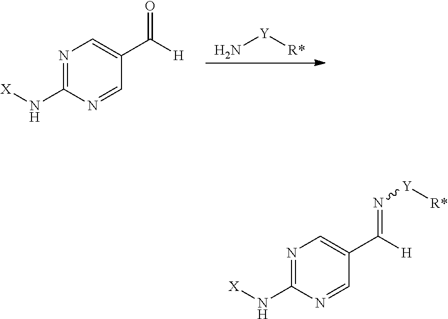

The compound of Scheme(I) reacts readily with alkoxyamine or hydrazine compounds as shown above to form an imine or (Y═O) hydrazide (Y═NH). This step provides a reasonably stable product, but the reaction is reversible. However, as is well known, the condensation product can be reduced by known methods: reducing the carbon-nitrogen double bond provides a stable product that can be isolated or further manipulated.

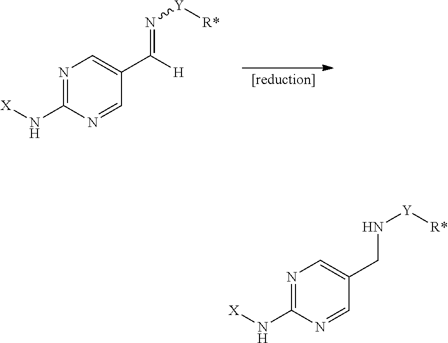

Linker L¹ or L² can be any convenient structure that can be used to connect the target molecule Tm to the structures depicted herein. The linker can comprise an alkyl chain segment, optionally interrupted with one or more heteroatoms (N, O or S), e.g. up to three such heteroatoms in a 6-atom segment, and/or one or two rings, for example phenyl and/or 3-6 atom cycloalkyl and/or 5-10 membered heterocyclyl or heteroaryl having one to three heteroatoms selected from N, O and S as ring members, such as pyridine, piperidine, triazole, and the like. Frequently, for ease of constructing the conjugates, the linker may include functional groups such as amide, carbamate, or sulfonamide, and substructures formed by reaction of a complementary pair of biorthogonal reaction handles, such as 1,2,3-triazoles. Selection and construction of various linkers is well within the skill level of the ordinary practitioner.

Suitable linkers for L¹ include [═N]—(CH₂)₀₋₁₀-Cy-(CH₂)₀₋₆—W, where [═N] represents the point where the linker is connected to ═N— in the Scheme being described, Cy represents a 5-6 membered ring such as phenyl, pyridinyl, cyclopropyl, and the like, and W represents a target compound or a biorthogonal reaction handle that is useful to connect the linker to a target compound. In some embodiments, one or more of the CH₂ groups can be replaced by a heteroatom, usually oxygen; for example, an alkylene chain of the formula (CH₂)₃ can be replaced by —CH₂CH₂O—.

Suitable linkers for L² include [Y]—(CH₂)₀₋₁₀-Cy-(CH₂)₀₋₆—W, where [Y] represents the point where the linker is connected to Y in the Scheme being described, Cy represents a 5-6 membered ring such as phenyl, pyridinyl, cyclopropyl, and the like, and W represents a target compound or a biorthogonal reaction handle that is useful to connect the linker to a target compound. In some embodiments, one or more of the CH₂ groups in the general formula can be replaced by a heteroatom, usually oxygen; for example, an alkylene chain of the formula (CH₂)₃ can be replaced by —CH₂CH₂O—.

The peptide may contain one or more post-translational modifications. A post-translational modification (PTM) of a peptide, polypeptide, or protein may be a covalent modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation (e.g., N-linked, O-linked, C-linked, phosphoglycosylation). A post-translational modification includes modifications of the amino terminus and/or the carboxyl terminus of a peptide, polypeptide, or protein. Post-translational modification can regulate a protein's “biology” within a cell, e.g., its activity, structure, stability, or localization. Phosphorylation is the most common post-translational modification and plays an important role in regulation of protein, particularly in cell signaling. The attachment of lipids to proteins enables targeting to the cell membrane. A post-translational modification can also include peptide, polypeptide, or protein modifications to include one or more detectable labels.

Optionally, the target molecule in the methods herein can be immobilized on a solid support (also referred to as “substrate surface”). The solid support can be any porous or non-porous support surface.

Proteins, polypeptides, or peptides can be immobilized to a surface of a solid support by its C-terminus, N-terminus, or an internal amino acid, for example, via an amine, carboxyl, or sulfhydryl group. Standard activated supports used in coupling to amine groups include CNBr-activated, NHS-activated, aldehyde-activated, azlactone-activated, and CDI-activated supports. Standard activated supports used in carboxyl coupling include carbodiimide-activated carboxyl moieties coupling to amine supports. Cysteine coupling can employ maleimide, iodoacetyl, and pyridyl disulfide activated supports. An alternative mode of peptide carboxy terminal immobilization uses anhydrotrypsin, a catalytically inert derivative of trypsin that binds peptides containing lysine or arginine residues at their C-termini without cleaving them.

The methods of the invention provide mild conditions for forming a conjugate linking a peptide to a target molecule by modifying an arginine residue of the peptide. While the Arnold Salt reacts irreversibly with guanidine, it can also react with amine groups; thus, in some embodiments, free amine groups including the terminal amine and any lysine side chains of the peptide may be protected before reacting it with an Arnold Salt.

The reaction medium typically comprises a buffer such as those disclosed herein, to provide a pH between about 8 and 12, including MOPS (3-(N-morpholino)propanesulfonic acid), HEPES, potassium phosphate, sodium phosphate, potassium biphosphate, sodium biphosphate, SSC (saline sodium citrate), CBC (sodium carbonate/bicarbonate), sodium carbonate, potassium carbonate, PIPES (piperazine-N,N′-bis(2-ethanesulfonic acid), PBS (phosphate-buffered saline), sodium pyrophosphate, TAPS ([tris(hydroxymethyl)methylamino]propanesulfonic acid), DAP (diammonium phosphate), CAPS (N-cyclohexyl-3-aminopropanesulfonic acid), sodium bicarbonate, potassium bicarbonate, sodium borate, sodium borate decahydrate, imidazole, and combinations of these that provide the desired pH. In some embodiments, the buffer is selected from bicarbonate and carbonate/bicarbonate.

The buffer is typically used at a concentration of 0.01 M or higher, often at a buffer concentration of 0.05 M or higher, and optionally at a buffer concentration of 0.1 M to 0.2 M, or higher than 0.2 M. In some embodiments of the invention, a buffer concentration of 1-2 M is used.

Reaction temperature can be about ambient temperature, i.e., 20° C. or 25° C., or it can be elevated to 30-100° C. to promote the irreversible reaction of the guanidine group with the Arnold Salt. Commonly, the reaction temperature can be about 40° C. or 50° C. or higher, and in some embodiments the reaction temperature is between about 50° C. and 80° C.

### EXAMPLES

The following examples are offered to illustrate but not to limit the methods, compositions, and uses provided herein. Certain aspects of the present invention, including, but not limited to, embodiments for ProteoCode™ peptide analysis assay, methods for attachment of nucleotide-peptide conjugate to a support, methods of making nucleotide-peptide conjugates, methods of generating barcodes, methods of analyzing extended recording tags to analyze a component of a peptide analyte were disclosed in earlier published applications US 2019/0145982 A1, US 2020/0348308 A1, US 2020/0348307 A1, US 2021/0214701 A1, US 2022/0049246 A1, and US 2022/0144885 A1, the contents of which are incorporated herein by reference in their entireties.

### Example 1. Assessment of Conditions for Arnold Salt Arginine Modification for a Model Peptide

This example describes the assessment of various conditions including buffers, pH, and temperature for Arnold Salt modification of peptides with at least one Arg amino acid residue. Unless otherwise specified, the Arnold Salt prepared as in Example 5 was used for the Examples.

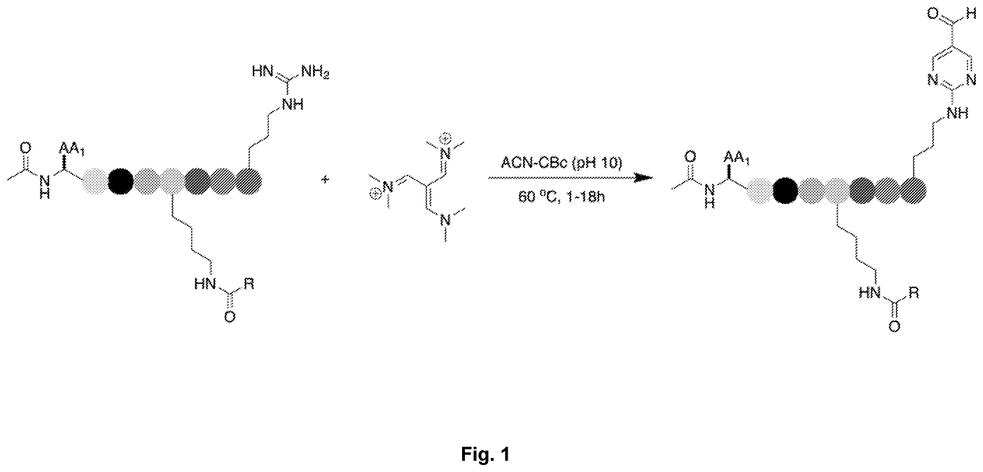

Methods for linking a CHD-containing conjugation reagent to an arginine residue in a peptide are known, but require harsh conditions, such as pH about 13. Milder conditions are needed to make this reaction useful in the context of complex molecules and biochemical mixtures, including in the presence of nucleic acids. Some milder conditions were disclosed in in the U.S. application Ser. No. 17/535,516 filed Nov. 24, 2021, but the reaction yield of CHD-Arg conjugation under neutral pH is below 50%. Thus, conditions and yield for Arnold Salt-Arg conjugation have been tested. See FIG. 1.

An array of buffers (0.01-0.5M) with a pH ranging from slightly basic to basic (pH 8-12) were tested. Two levels of reaction temperature (37° C. and 60° C.) were initially tested. To quantitatively determine the reaction yield, an arginine-free peptide was introduced to the CHD/arginine peptide mixture as an internal control for LCMS analysis. A test peptide Ac-K(DTB)GVAMPGAEDDVVA [symbol] , SEQ ID NO: 1, has been N-terminally acetylated and pre-treated with Desthiobiotin (DTB) to block reaction of the Arnold salt reagent with primary amines on N-terminus and Lys residues. For this purpose, EZ-Link™ NHS-Desthiobiotin (Thermo Fischer) was used according to manufacturer's instructions. Briefly, a final concentration of 10 mM NHS-desthiobiotin was used, and the mixture was incubated for 1 hour at 60° C. Then, one volume of 1 M Tris, pH 7.4 was added to quench excess, unreacted NHS.

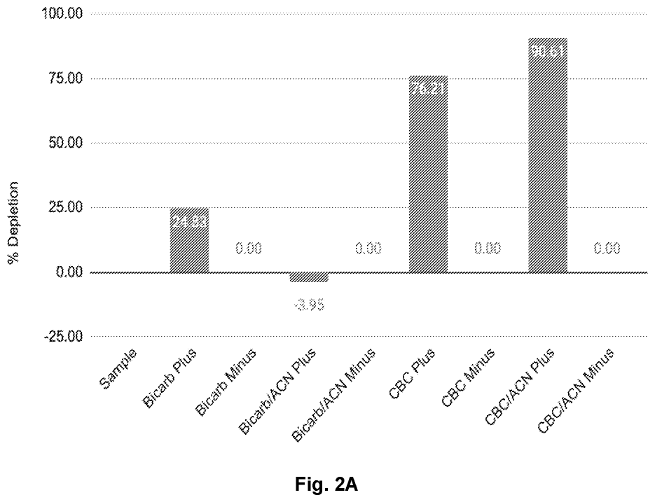

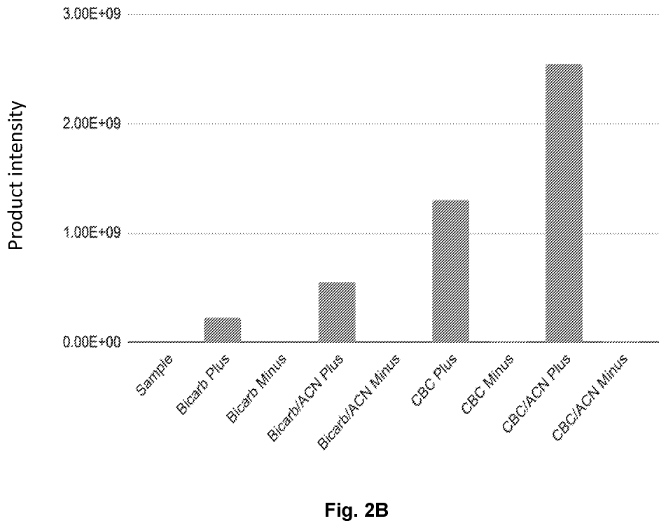

Exemplary buffer conditions tested include 50 mM sodium bicarbonate buffer, pH=8.5, and 0.1 M Carbonate/Bicarbonate buffer, pH 10.0, both optionally supplemented with 50% Acetonitrile (ACN), see FIG. 2A-B. Results shown on FIG. 2A-B are for reactions that were incubated at 60° C. for 18 h, with or without the 50 mM Arnold Salt reagent. The results of Arnold Salt-Arg conjugation have been evaluated by two parameters based on LC-MS run: 1) Percent depletion (FIG. 2A) shows depletion of the initial peptide peak based on the LC chromatographic data; and 2) Product intensity was measured by ion counts in the LC-MS runs of the resulting product, which is also detected on the LC chromatogram (FIG. 2B). Before the Arnold salt reagent addition, the mass of the peptide modified with Ac and DTB was m/z 876.94 (+2), whereas after reaction with the Arnold Salt reagent, the expected mass of the modified peptide was m/z 908.94 (+2). The peptides before and after reactions were purified using C18 spin columns, and were diluted to 0.5 uM right before the injection to the LC-MS/MS system (Orbitrap Exploris 240 Mass Spectrometer, Thermo Fisher).

FIG. 2A-B indicate that incubation of the peptide in 0.1 M Carbonate/Bicarbonate buffer, pH 10.0 with 50 mM Arnold salt at 60° C. for 18 h resulted in more than 70% conversion to the Arnold Salt-peptide reaction product, which was evident by disappearance of the initial peptide peak, and appearance of the modified peptide peak. The proper Arnold Salt-Arg conjugation in the peptide was confirmed by MS/MS data, which showed the presence of “y” ions corresponding to the modified peptide having m/z 908.9 (+2). Addition of 50 ACN during the reaction with Arnold Salt further increased yield of the Arnold Salt-Arg conjugation reaction above 90% (see, e.g., FIG. 2A-B).

Further, selected reaction conditions (0.1 M Carbonate/Bicarbonate buffer, pH 10.0, 50 mM Arnold Salt at 60° C. for 18 h) did not significantly affect stability of several tested post-translational modifications (PTMs) of the peptide. In particular, Arnold Salt conditions protect p-Thr from beta-elimination, and p-Tyr was not affected by incubation with the abovementioned Arnold Salt condition. Also, p-Ser was not beta-eliminated. For this experiment, 2 nmol of peptides containing p-Ser and p-Thr were incubated with or without 100 mM Arnold Salt in 0.1 M Carbonate/Bicarbonate buffer with 50% ACN, pH 10.0, at 60° C. for 18 h, and potential mass shifts of the modified peptides due to beta-elimination were evaluated by LC-MC/MC (data not shown).

### Example 2. Assessment Efficiency of Arnold Salt Arginine Modification at the Proteome Level (In Human Serum Samples)

The conditions for Arnold Salt-Arg conjugation selected in Example 1 were applied to human serum samples. Briefly, about 100 ug of human serum samples (and Top 14 depleted serum aliquots of those, prepared using High Select Top14 Abundant Protein Depletion Resin, Thermo Fisher, which can deplete greater than 95% of the 14 most abundant proteins in serum) were subjected to denaturation and digestion as follows. For a 10-μg serum sample, the sample was diluted to the desired protein input concentration in NHS-DTB (N-hydroxysuccinimide-desthiobiotin) buffer (10 ug/45 μL; 100 mM Carbonate/Bicarbonate buffer pH 9, 2% sodium deoxycholate (SDC)). 0.5 M TCEP (stock solution) was added for a final concentration of 5 mM TCEP. Samples were incubated for 15 min at 37° C. After cooling, sufficient 0.5 M iodoacetamide (IAA) stock was added for a final concentration of 20 mM. Samples were incubated at 37° C. for 15 min to allow the alkylation to proceed, then 100 mM NHS-DTB stock was added to each sample for a final concentration of 10 mM NHS-desthiobiotin, and incubated for 1 hour at 60° C. One volume of 1 M Tris, pH 7.4 was added to quench excess of unreacted NHS. Trypsin was added at a 1:25 ratio, by mass, for each sample and incubated for 2 hrs at 37° C. to digest the sample. Acidification Solution (50% acetonitrile, 2% formic acid in high purity water) was added and the samples were centrifuged to pellet insoluble material (precipitated SDC) and the supernatant was kept. About 200 μL of digested protein sample congaing about 100 ug of peptides was purified from salts and excess reagents using PreOmics® PHOENIX™ column. Other SCX (strong cation exchange) columns, SEC (size-exclusion chromatography) columns, SPE (solid-phase extraction) columns, centrifugal filters, desalting columns, reverse phase LC, physisorption methodologies, and other suitable scavenger beads/resins can also be used for peptide purification.

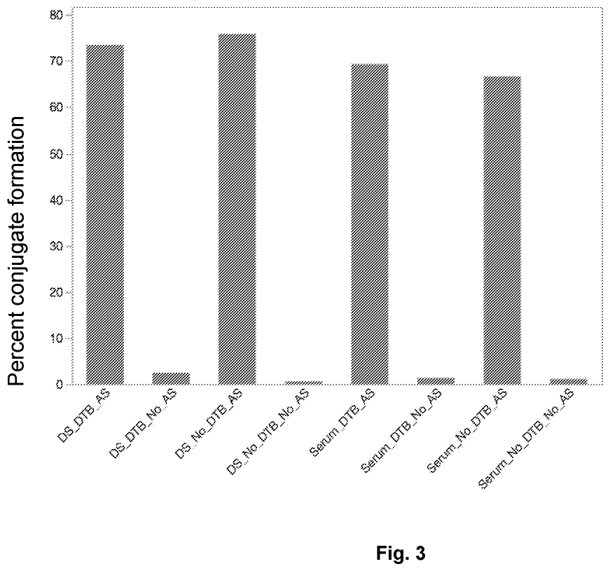

About 100 ug of purified peptides from the treated samples were subjected to the Arnold Salt modification. The Arnold Salt reaction conditions were similar to the conditions selected in the Example 1: 0.1 M Carbonate/Bicarbonate buffer, pH 10.0 with 50% ACN, 100 mM Arnold Salt at 60° C. for 18 h. The quenched samples were cleaned up with Preomics or C18 spin columns and subjected to LC-MS/MS analysis. LC-MS/MS analysis showed 60-70% conversion yield for the Arnold Salt-Arg conjugation in proteins across the human serum proteome (FIG. 3).

### Example 3. Arnold Salt-Modified Peptides React Rapidly and Efficiently with an Aldehyde-Reactive Moiety with About 100% Conversion

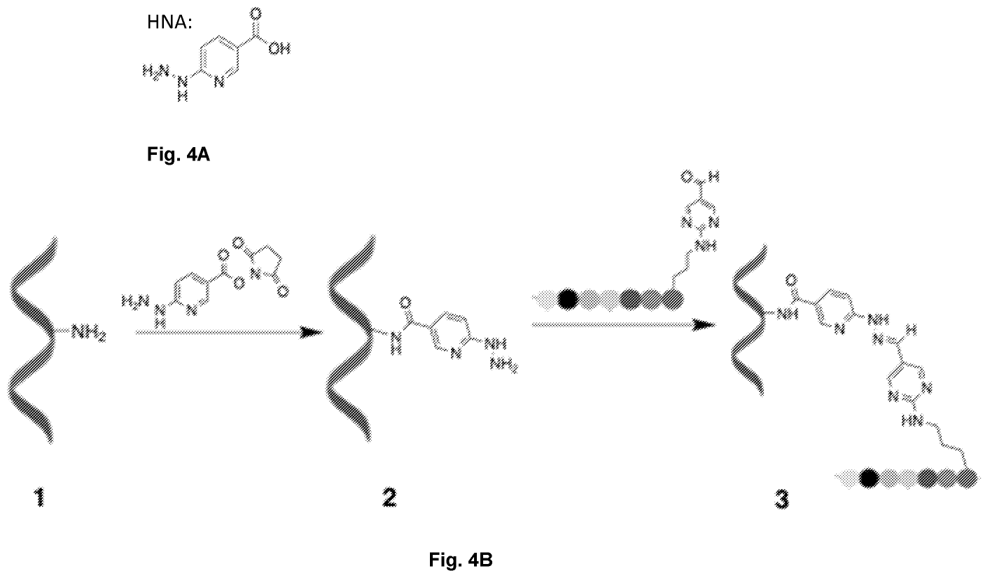

Utility of peptides having Arg residues modified with Arnold Salt can be shown in a reaction with Hydrazino nicotinamide (HNA, see FIG. 4A)-modified nucleic acids (see compound 2 in FIG. 4B) or other target macromolecules. As a first step, Arnold Salt-modified peptides can react with an HNA moiety with about 100% conversion rate in 1 h. 20 nmol of an Arnold Salt-modified peptide were reacted with 40 nmol of HNA in a citrate buffer (pH=6.5) for 1 h at room temperature. The expected conjugated product having an increased mass of 134 Da based on the formed structure shown in compound 3 of FIG. 4B was detected by LC-MS/MS. Rapid disappearance of the peak corresponding to the unreacted Arnold Salt-modified peptide indicated efficiency of the reaction (>99% conversion within 1 h at room temperature).

This reaction allows for forming conjugates of Arnold Salt-modified peptides with target moieties modified with HNA. FIG. 4B shows an example of forming a conjugate between a peptide and a nucleic acid modified with HNA (the nucleic acid in this Example is functionalized with a primary amine at a specific place, suitable for conjugation with NHS-HNA).

### Example 4. Evaluating Efficiency of On-Bead Modification of a Peptide with Arnold Salt Reagent

In this example, the reaction yield for modifying Arg residue of a peptide immobilized on a solid support (a porous NHS-Activated sepharose bead, Cytiva, USA) with Arnold Salt reagent was estimated. A test peptide [Ac]-K(N3)GGGAENLYFQSAEWHIFAR, SEQ ID NO: 2, has a pre-installed azide on Lys residue and an N-terminal acetyl group. The peptide was immobilized on DBCO sepharose beads using a click chemistry reaction between DBCO and azide. The peptide contained a specific site for TEV protease cleavage located after Q residue. 20 nmol of the immobilized peptide on beads were treated with 100 mM Arnold Salt in 0.1 M Carbonate/Bicarbonate buffer, pH 10.0, at 60° C. for 18 h. The same amount of the immobilized peptide on beads were used as a control (incubated under identical conditions in 0.1 M Carbonate/Bicarbonate buffer without Arnold Salt). After 18 h, the reactions were quenched with 5M GdmCl, and beads were washed twice to remove residual reagents. Then, 450 Units of TEV protease were added to both treated and non-treated peptides on beads and incubated for 1.5 hrs to cleave and release C-terminal portions of the immobilized peptide (SAEWHIFAR, SEQ ID NO: 3). Released peptides were purified using C18 columns and analyzed by Lc-MS/MS to determine percent conversion of R residues. Expected mass of the unmodified released peptide was m/z=558.29 (+2), while expected mass of the released peptide modified with Arnold Salt was m/z=590.29 (+2).

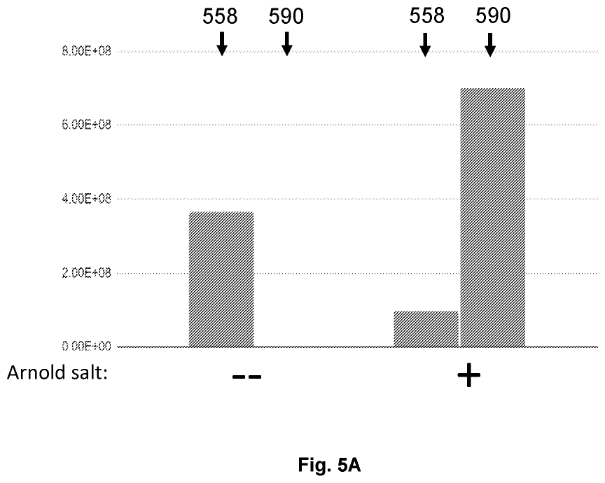

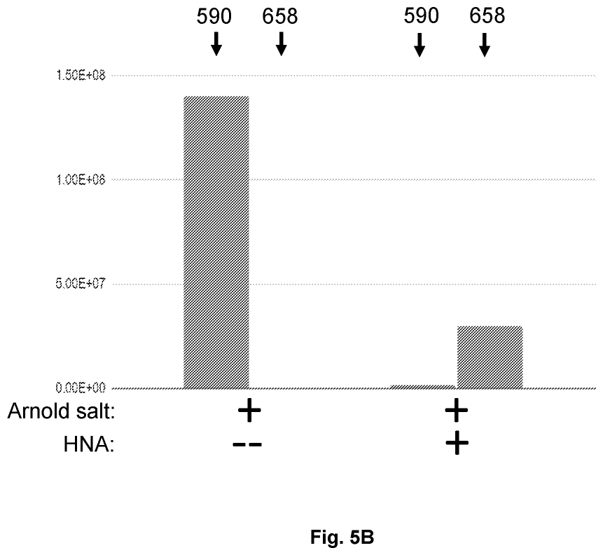

Product intensities were measured by ion counts in the LC-MS runs of the resulting products (FIG. 5A). In control reactions, only unmodified released peptide was detected (m/z=558.29 (+2)), while in reactions treated with Arnold salt, both unmodified and modified released peptides were detected, with appr. 73% conversion to the Arnold Salt-peptide reaction product estimated based on the ion count (FIG. 5A).

Additionally, a similar experiment was performed where, after reaction with Arnold Salt, the immobilized peptides on beads were further incubated with 40 nmol of HNA (see FIG. 4A) in a citrate buffer (pH=6.5) for 1 h at room temperature. As a control, the immobilized peptides on beads were incubated under identical conditions in a citrate buffer without HNA. Then, the immobilized peptides on beads were processed as described above (cleavage with TEV protease, purification and LC-MS/MS analysis). Expected mass of the released peptide in the reaction without HNA was m/z=590.29 (+2), while expected mass of the released peptide in the reaction with HNA was m/z=657.81 (+2). Product intensities were again measured by ion counts in the LC-MS runs of the resulting products, indicating about 99% conversion for reaction of the Arnold Salt-peptide reaction products with HNA (FIG. 5B).

### Example 5. Synthesis of bis(hexafluorophosphate) Arnold Salt

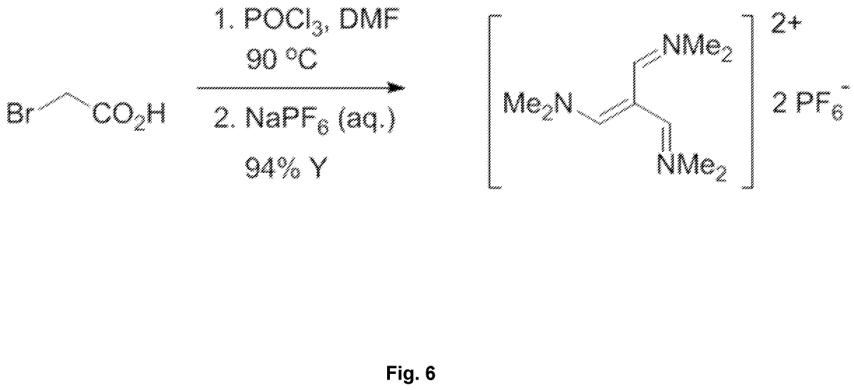

This procedure was adopted from Maltsev et al, Eur. J. Org. Chem. 2014, 7426 (and the references therein on Org prep daily blog), see, e.g., FIG. 6.

Anhydrous DMF (160 mL) in a 0.5 L round flask was cooled in an ice bath and neat POCl₃ 40 mL (436 mmol) was added gradually over a 10 min period (exothermic). The mixture was warmed up to RT, and bromoacetic acid solid 19.00 g (136.7 mmol, Aldrich 99%) was added quickly in one portion. The flask was equipped with a reflux condenser and the mixture was stirred under Ar in an oil bath at 90 C for 9 hours. The mixture turned orange, with a slow CO₂ evolution. The mixture was cooled to RT, DMF was distilled out on high vacuum (RT to 90 C at 0.5 Torr), and the obtained thick residue was treated with ice-cold water (100 mL) in a sonicator bath. An exothermic hydrolysis commenced. When all residue dissolved and the mixture cooled to RT, solid sodium hexafluorophosphate 46.2 g (275 mmol) was added in one portion followed by some additional water (50 mL). The mixture was shaken vigorously for 10 min, and the precipitated product was collected using a large Buchner funnel. The solid cake was compressed on the frit, washed with ice-cold water (5×20mL), dried by suction and then on high vacuum. The yield of bis(hexafluorophosphate) Arnold Salt was approximately 94%.

### Example 6. Peptide Sample Preparation Workflow for ProteoCode™ Peptide Analysis Assay

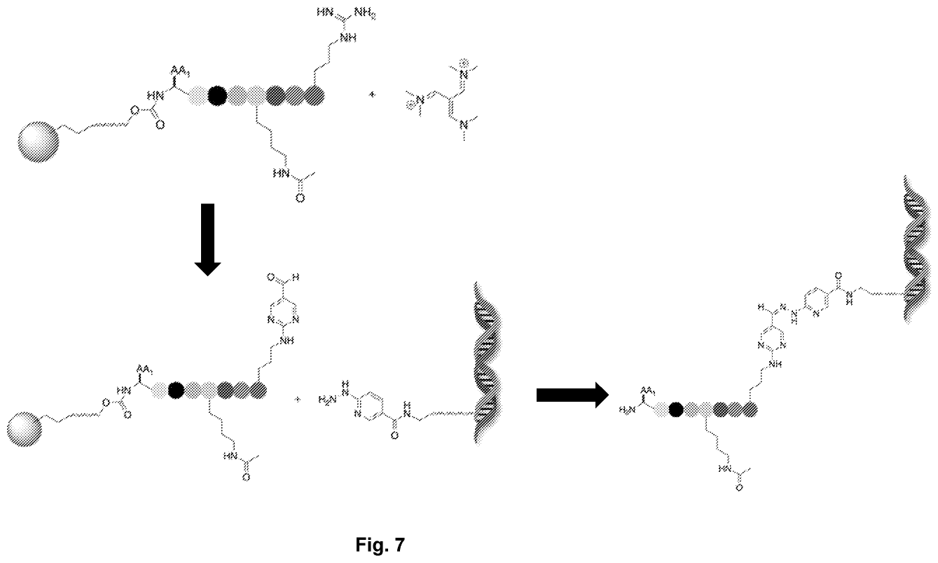

This example demonstrates an exemplary sample preparation workflow used for preparing peptides using the Arnold Salt-orthogonal handle (e.g., HNA). The exemplary workflow depicted in FIG. 7 outlines preparation of Arnold Salt-labeled and DNA-coupled peptides starting from an unpurified proteomic sample. This example also describes assessing and using the prepared peptides in a ProteoCode™ assay which utilizes DNA encoding.

### Protein Denaturation and Digestion:

For a 10 μg protein sample, the sample was diluted to the desired protein input concentration in NHS-DTB (N-hydroxysuccinimide-desthiobiotin) buffer (10 ug/45 μL; 100 mM Carbonate/Bicarbonate buffer pH 9, 2% sodium deoxycholate (SDC)). 0.5 M TCEP (stock solution) was added for a final concentration of 5 mM TCEP. Samples were incubated for 15 min at 37° C. After cooling, sufficient 0.5 M iodoacetamide (IAA) stock was added for a final concentration of 20 mM. Samples were incubated at 37° C. for 15 min to allow the alkylation to proceed, then 100 mM NHS-DTB stock was added to each sample for a final concentration of 10 mM NHS-desthiobiotin, and incubated for 1 hour at 60° C. One volume of 1 M Tris, pH 7.4 was added to quench excess, unreacted NHS. Trypsin was added at a 1:25 ratio, by mass, for each sample and incubated for 2 hrs. at 37° C. to digest the sample. Acidification Solution (50% acetonitrile, 2% formic acid in high purity water) was added and the samples were centrifuged to pellet insoluble material (precipitated SDC), and the supernatant was kept.

Purification of peptides: 200 μL digested protein sample was purified away from salts and excess reagents using PreOmics® PHOENIX™ columns.

Arnold Salt functionalization of C-terminal arginines was performed according to the conditions described in Examples 1 and 2. Each sample was incubated with 100 mM Arnold Salt in 0.1 M Carbonate/Bicarbonate buffer, pH 10.0 at 60° C. for 18 h. The excess of reagents was removed using PreOmics® PHOENIX™ columns. Then, peptides were joined to a solid support by using capture with Streptavidin beads. Streptavidin beads were prepared (washed 3× with PBS-T) and added to the samples with rotation for 2 h to allow for streptavidin bead binding. After the incubation period, samples were washed twice with 200 uL PBS-T and resuspended in 10 μL of 125 uM HNA-DNA in 0.2 M citrate buffer (pH=6.5). The samples were incubated with rotation at room temperature overnight (16-18 hours) to generate peptide-DNA conjugates (peptides with associated recording tags).

Sample Barcoding. Upon completion of incubation, beads were centrifuged and washed to remove any excess HNA-DNA. Sample barcodes were added and beads were washed twice with 200 μL PBS-T. The peptide-DNA conjugates were eluted with 10 μL 4 mM biotin, 20 mM Tris-HCl, and 50 mM NaCl. Conjugate formation and barcoding were confirmed by loading 0.5 μL of sample (5 pmol) on TBU gel electrophoresis. (15% TBU (TBE-Urea) gel, 200V, 50 min). Various peptides (e.g., protein based, some rationally designed for assay) were treated using this exemplary workflow. The peptides were then immobilized on a solid support (beads; NHS-Activated Sepharose High Performance, Cytiva, USA). The DNA of the peptide-DNA conjugates was hybridized and ligated to capture DNAs containing a complementary sequence attached to beads at appropriate spacing and density (as disclosed in US 20200348308 A1). Briefly, the capture DNAs were conjugated to the beads using trans-cyclooctene (TCO) and methyltetrazine (mTet)-based click chemistry. TCO-modified short hairpin capture DNAs (16 basepair stem, 4 base loop, 17 base 5′ overhang) were reacted with mTet-coated beads. Phosphorylated nucleic acid-polypeptide conjugates (20 nM) were annealed to the hairpin DNAs attached to beads in 0.5 M NaCl, 50 mM sodium citrate, 0.02% SDS, pH 7.0, and incubated for 30 minutes at 37° C. The beads were washed once with PBST (1× phosphate buffer, 0.1% Tween 20) and resuspended in 1× Quick ligation solution (New England Biolabs, USA) with T4 DNA ligase. After a 30 min incubation at 25° C., the beads with immobilized peptide-DNA conjugates were washed once with PBST, three times with 0.1 M NaOH, 0.1% Tween 20, three times with 1× phosphate buffer, 0.1% Tween 20, and resuspended in 50 μL of PBST. Peptide-DNA conjugates joined to the solid support were used in the following peptide analysis assay.

### ProteoCode™ Peptide Analysis Assay.

After the peptide-DNA conjugates prepared using the exemplary workflow described above were immobilized on a solid support, ProteoCode™ peptide analysis assay was performed as disclosed in US published patent applications US 20190145982 A1 and US 20210214701 A1, incorporated herein. In the assay, N-terminal amino acid (NTAA) residues of peptides from the peptide-DNA conjugates joined to the solid support (peptides with associated DNA recording tags) were functionalized by an N-terminal modification specific for recognizing binding agents. The immobilized and functionalized peptide-DNA conjugates were contacted with binding agents each conjugated with a nucleic acid coding tag containing identifying information regarding the associated binding agent. Binding agents configured to recognize chemically modified N-terminal amino acid (NTAA) residues used herein were disclosed in U.S. patent application Ser. No. 17/539,033, filed on Nov. 30, 2021; and in PCT patent application PCT/US2021/065798, filed on Dec. 30, 2021. Binding agents were used simultaneously as a set, and altogether have specificity for most of the modified NTAA residues. If a binding agent binds its cognate modified NTAA residue of the peptide, and affinity of the binding agent to the immobilized peptide is strong enough (typically, Kd should be less than 500 nM, and preferably, less than 200 nM), the coding tag associated with the binding agent and the recording tag associated with the peptide form hybridization complex via hybridization of the corresponding spacer regions to allow transfer of identifying information from the coding tag to the recording tag via a primer extension reaction (referred to as the “encoding reaction”), generating extended recording tag.

Following binding of the binding agent to the functionalized NTAA of the immobilized peptide and the encoding reaction, the functionalized NTAA is cleaved to expose a new NTAA residue of the immobilized peptide. In preferred embodiments of the disclosed methods, cleaving the functionalized NTAA residue of the peptide is done by an engineered enzyme, such as an engineered dipeptidyl aminopeptidase disclosed in the published patent applications US 2021/0214701 A1 and WO 2021/141924 A1, incorporated herein.

In some particularly preferred embodiments of the disclosed methods, cleaving the functionalized NTAA residue of the peptide is done by an engineered enzyme, such as a modified cleavase, which is configured to cleave a peptide bond between a terminally labeled amino acid residue and a penultimate terminal amino acid residue of a polypeptide, wherein the modified cleavase is derived from a dipeptidyl aminopeptidase, which removes an unlabeled terminal dipeptide from a polypeptide, wherein the dipeptide aminopeptidase comprises an amino acid sequence having at least 20% sequence identity to the amino acid sequence of SEQ ID NO: 4 and also comprising an asparagine residue at a position corresponding to position 191 of SEQ ID NO: 4, a tryptophan residue or phenylalanine residue at a position corresponding to position 192 of SEQ ID NO: 4, an arginine residue at a position corresponding to position 196 of SEQ ID NO: 4, an asparagine residue at a position corresponding to position 306 of SEQ ID NO: 4, and an aspartate residue at a position corresponding to position 650 of SEQ ID NO: 4; and wherein the modified cleavase comprises two or more amino acid substitutions in the residues corresponding to positions N191, W/F192, R196, N306, and D650 of SEQ ID NO: 4, as disclosed in in the patent application US 20210214701 A1.

In some particularly preferred embodiments of the disclosed methods, the modified cleavase used for cleaving the functionalized NTAA residue of the peptide comprises amino acid sequence that is at least 80% homologous to sequence set forth in SEQ ID NO: 5.

Following the cleavage of the functionalized NTAA from the immobilized peptide, the encoding reaction is repeated one or more times, comprising: functionalizing the newly exposed NTAA residue; binding of the binding agent with a coding tag to the newly functionalized NTAA of the immobilized peptide; and following binding, transferring identifying information from the coding tag to the extended recording tag associated with the immobilized peptide.

Three cycles of encoding (information transfer from coding tags to recording tags) with two elimination cycles in between were performed. Elimination of the NTAA exposed a new NTAA available for recognition by a binding agent provided in the next cycle. Sequencing of extended recording tags after one or more encoding cycles was used to identify binding agent(s) that was(were) bound to the immobilized peptide, providing structural information regarding the immobilized peptide. Estimating fractions of the recording tags being extended (encoded) during primer extension reaction provided estimates of efficiency of the encoding reactions, which directly correlates with binding affinity of the binder to the peptide. After completion of the binding, encoding, functionalization and elimination cycle(s), the extended recording tags were capped with an adapter sequence, subjected to PCR amplification, and analyzed by next-generation sequencing (NGS). In summary, peptides from biological samples modified using the Arnold Salt reagent and conjugated to nucleic acid (DNA) recording tags according to the exemplary workflow shown in FIG. 7 can be successfully analyzed by the ProteoCode™ peptide analysis assay.

Using the methods in these examples and general knowledge in the field, a wide array of conjugation reactions and conjugates of the invention can be practiced with various reactive handles, target molecules, detectable labels, and binding agents.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method to functionalize a guanidine on the side chain of an amino acid residue, wherein the method comprises contacting the amino acid residue with an Arnold Salt under conditions that result in formation of a formyl-substituted pyrimidine of Formula (I): 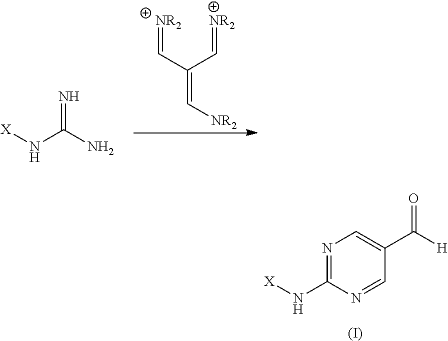
wherein:
X represents the remainder of the side chain of the amino acid residue, and
each R is independently selected from methyl, ethyl, and propyl, or two R on the same N can optionally be taken together with the nitrogen to which they are attached to form a 4-6 membered ring.

2. The method of claim 1, wherein the amino acid residue is an arginine in a peptide.

3. The method of claim 1, wherein the arginine residue is contacted with the Arnold Salt in a medium comprising an aqueous buffer and further comprising an organic cosolvent.

4. The method of claim 1, wherein the arginine residue is contacted with the Arnold Salt in a medium at a temperature between 40° C. and 80° C.

5. The method of claim 4, wherein the medium has a pH between 8 and 12.

6. The method of claim 1, wherein at least 50% of the arginine residue in a sample is modified to form the formyl pyrimidine of Formula (I).

7. The method of claim 1, wherein the Arnold Salt is associated with a counterion selected from Br₃⁻, ClO₄⁻, PF₆⁻, and BF₄⁻.

8. The method of claim 2, wherein the peptide is joined to a solid support.

9. The method of claim 1, which further comprises a step of contacting the formyl pyrimidine of Formula (I) with a compound that comprises a target molecule and an aldehyde-reactive reaction handle having an —NH₂ group, to form a conjugate of the formula: 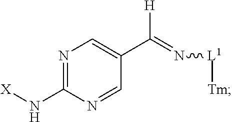
wherein X represents the alkyl portion of the side chain of an arginine residue,
L¹ represents a linker, and
Tm represents the target molecule.

10. The method of claim 9, wherein the conjugate is of the formula: 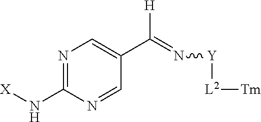
wherein X represents the alkyl portion of the side chain of the arginine residue,
Y is O or NH,
L² represents a linker; and
Tm represents the target molecule.

11. The method of claim 10, wherein the method further comprises a step of contacting the conjugate with a reducing agent to provide a conjugate of the formula: 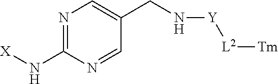
wherein X represents the alkyl portion of the side chain of the arginine residue,
Y is O or NH, and
Tm represents the target molecule.

12. The method of claim 9, wherein X represents a peptide connected to the remainder of the conjugate through the alkyl portion of the side chain of an arginine residue of the peptide.

13. The method of claim 12, wherein Tm represents a polynucleotide.

14. A modified amino acid residue comprising a substructure of Formula (I): 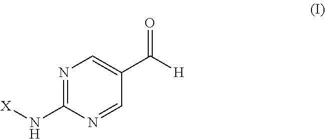
wherein X represents the side chain portion of the amino acid residue.

15. The modified arginine residue of claim 14, wherein the amino acid residue is an arginine in a peptide.

16. The modified arginine residue of claim 15, wherein the peptide is joined to a solid support.

17. A conjugate comprising the structure: 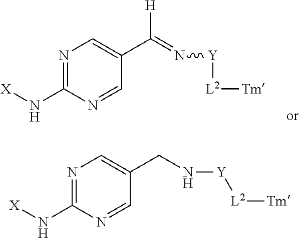
wherein X represents a side chain of an amino acid residue,
L² is a linker;
Y is selected from a bond, O and NH; and
Tm′ represents a target molecule.

18. The conjugate of claim 17, wherein X represents the side chain of an arginine residue in a peptide.

19. The conjugate of claim 18, wherein the peptide is joined to a solid support.

20. The conjugate of claim 18, wherein Y is —O— or —NH—.

21. A method of attaching a peptide comprising at least one arginine residue to a target molecule, the method comprising the steps of:
(a) contacting the at least one arginine residue with an Arnold Salt under conditions that result in formation of a formyl-substituted pyrimidine of Formula (I): 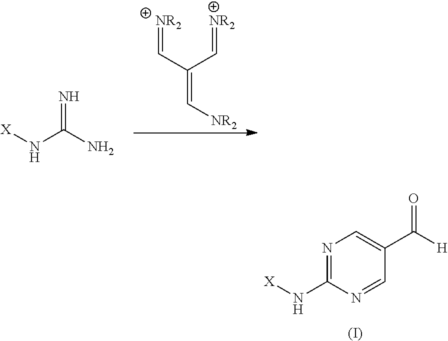
wherein:
X represents the peptide connected through the at least one arginine residue, and each R is independently selected from methyl, ethyl, and propyl, or two R on the same N can optionally be taken together with the nitrogen to which they are attached to form a 4-6 membered ring; and
(b) contacting the formyl pyrimidine of Formula (I) with a compound that comprises the target molecule and an aldehyde-reactive reaction handle having an —NH₂ group, to form a conjugate of the formula: 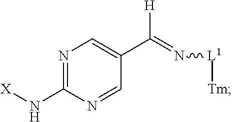
wherein X represents the peptide connected through the at least one arginine residue, L¹ represents a linker, and Tm represents the target molecule.

22. The method of claim 21, wherein the target molecule comprises a polynucleotide.

23. The method of claim 21, wherein the conjugate is of the formula: 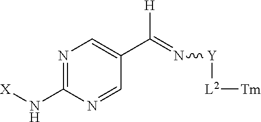
wherein X represents the alkyl portion of the side chain of the arginine residue,
Y is —O— or —NH—, L² represents a linker; and Tm represents the target molecule.


SEQUENCE LISTING (5 sequences)

SEQ ID NO: 1 - protein, 15 aa, synthetic construct
  REGION 1..15: note = test peptide
  SITE 1: note = MISC_FEATURE - N-terminus acetyl and Desthiobiotin
KGVAMPGAEDDVVAR

SEQ ID NO: 2 - protein, 20 aa, synthetic construct
  REGION 1..20: note = test peptide
  SITE 1: note = MISC_FEATURE - N-terminus acetyl and azide
KGGGAENLYFQSAEWHIFAR

SEQ ID NO: 3 - protein, 9 aa, synthetic construct
  REGION 1..9: note = test peptide
SAEWHIFAR

SEQ ID NO: 4 - protein, 700 aa, Thermomonas hydrothermalis
  REGION 1..700: note = Dipeptidyl peptidase from Thermomonas hydrothermalis (without the signal peptide)
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 5 - protein, 723 aa, synthetic construct
  REGION 1..723: note = an engineered cleavase
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMGMWPTHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNRTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFAGTLESVASNWVFDPVLTRMISVDQRYMRWIMQEVMPAPQLLEELGVPPRQ